(19) United States
(12) Patent Application Publication
(10) Pub. No.: US 2005/0202463 A1
(43) Pub. Date: Sep. 15, 2005

(54) Modulators of dimethylarginine dimethylaminohydrolase and methods of use thereof

(76) Inventors: John P. Cooke, Palo Alto, CA (US); Ken Y. Lin, Boston, MA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/986,738

(22) Filed: Nov. 12, 2004

Related U.S. Application Data
(60) Provisional application No. 60/520,538, filed on Nov. 14, 2003.

(51) Int. Cl. C12Q 1/68

(52) U.S. Cl. 435/6

(57) ABSTRACT

The present invention provides methods of identifying agents that increase dimethylarginine dimethylaminohydrolase (DDAH) levels and/or activity. The present invention further provides agents that increase DDAH levels and/or activity, and compositions comprising the agents. The invention further provides methods of treating insulin resistance, and methods of lowering blood glucose levels in an insulin-resistance individual, the methods generally involving administering to the individual an agent that increases DDAH levels and/or activity. The invention further provides methods of treating conditions in which plasma or tissue ADMA levels are elevated, by increasing the activity or expression of DDAH.

### CROSS-REFERENCE

This application claims the benefit of U.S. Provisional Patent Application No. 60/520,538, filed Nov. 14, 2003, which application is incorporated herein by reference in its entirety.

### STATEMENT REGARDING FEDERALLY SPONSORED RESEARCH

The U.S. government may have certain rights in this invention, pursuant to grant nos. 5RO1 HL63685 awarded by the National Institutes of Health.

### FIELD OF THE INVENTION

The present invention is in the field of modulators of dimethylarginine dimethylaminohydrolase, and methods of reducing insulin resistance.

### BACKGROUND OF THE INVENTION

Insulin resistance is a condition in which the tissues of the body fail to respond normally to insulin, and is an important metabolic abnormality in Type 2 diabetes. Insulin-resistant individuals are at significantly increased risk of developing cardiovascular disease and atherosclerosis.

It has been previously reported that asymmetric dimethylarginine (ADMA) levels are elevated in patients with insulin resistance. Stuhlinger et al. (2002) J. Am. Med Assoc. 287:1420-1426. Stuhlinger concluded that insulin resistance increases ADMA levels. Others have come to the same conclusion. Nash (2002) J. Am. Med. Assoc. 287:1451. The study reported in Lin et al. ((2002) Circulation 106:987-992) is based on the assumption that diabetes mellitus (DM) increases ADMA, a view which has been reported elsewhere. Abbasi et al. (2001) Am. J. Cardiol. 88:1201-1203. Lin concluded that glucose-induced impairment of dimethylarginine dimethylaminohydrolase (DDAH), and enzyme that degrades ADMA, causes ADMA accumulation and may contribute to endothelial vasodilator dysfunction in DM. Certain drugs, such as thiazolidinedione, have been reported to lower ADMA levels (Stuhlinger et al. (2002), supra); however, the mechanism of action of thiazolidinedione is unknown. Current dogma is that thiazolidinediones act as agonists of a transcriptional factor Peroxisome Proliferator-Activated Receptor (PPAR), the activation of which leads to increased expression of the insulin-dependent glucose transporter GLUT-4. However, it has been suggested that the thiazolidinediones could possibly work by reducing oxidative stress, increasing DDAH activity, and/or increasing ADMA degradation by some other means. Chan and Chan (2002) Diabetologia 45:1609-1616. However, any effect of thiazolidinedione on DDAH activity is speculative. Others have indicated that the reported effects of thiazolidinediones are inconclusive, and that the mechanism by which thiazolidinediones improve insulin sensitivity are poorly understood. Wheatcroft et al. (2003) Diabetic Medicine 20:255-268. Similarly, while metformin has been shown to decrease circulating ADMA levels, its effect has been proposed to be through stimulation of AMP-activated protein kinase. Wheatcroft et al. (2003), supra.

The currently available drugs used to treat insulin resistance include thiazolidinediones, biguanides, and alpha-glucosidase inhibitors. Despite the availability of such agents, there is an ongoing need in the art for agents that treat insulin resistance. The present invention addresses this need.

Literature

Lin et al. (2002) Circulation 106:987-992; Stuhlinger et al. (2002) J. Am. Med. Assoc. 287:1420-1426; Chan and Chan (2002) Diabetologia 45:1609-1616; Wheatcroft et al. (2003) Diabetic Med. 20:255-268; Nash (2002) J. Am. Med. Assoc. 287:1451-1452; Achan et al. (2002) Circ. Res. 90:764-769.

### SUMMARY OF THE INVENTION

The present invention provides methods of identifying agents that increase dimethylarginine dimethylaminohydrolase (DDAH) levels and/or activity. The present invention further provides agents that increase DDAH levels and/or activity, and compositions comprising the agents. The invention further provides methods of treating insulin resistance, and methods of lowering blood glucose levels in an insulin-resistance individual, the methods generally involving administering to the individual an agent that increases DDAH levels and/or activity. The invention further provides methods of treating conditions in which plasma or tissue ADMA levels are elevated, by increasing the activity or expression of DDAH.

### BRIEF DESCRIPTION OF THE DRAWINGS

FIG. 1A depicts elevated levels of human DDAH protein expression in various tissues of the DDAH transgenic mouse, compared to the level in those tissues in a wild-type control mouse.

FIG. 1B depicts elevated DDAH activity in tissues of the DDAH transgenic (“DDAH Tg”) mouse compared to the level of DDAH activity in the wild-type control mouse, as shown in the left panel. Elevated DDAH activity is associated with a reduction in the plasma ADMA level, as shown in the right panel.

FIG. 2 depicts elevated levels of urinary nitrogen oxides (NOₓ) in the DDAH transgenic (“DDAH Tg”) mouse, revealing that increased DDAH activity is associated with increased NO synthesis.

FIG. 3 depicts the reduction in mean arterial pressure (MAP) and systemic vascular resistance (SVR) in the DDAH transgenic mouse.

FIG. 4 depicts plasma glucose concentration vs. time following intraperitoneal (i.p.) injection of insulin in wild-type mice treated with phosphate-buffered saline (PBS; “WT/PBS”) or asymmetric dimethylarginine (ADMA; “WT/ADMA”), and in DDAH transgenic mice treated with ADMA (“Transgenic/ADMA”).

### DEFINITIONS

The term “agent” includes any substance, molecule, element, compound, entity, or a combination thereof. The term “agent” includes, but is not limited to, e.g., small organic molecules; small inorganic molecules; and macromolecules such as polysaccharides, polynucleotides, polypeptides, glycoproteins, lipoproteins, and the like. An “agent” is a natural product, a synthetic compound, a semi-synthetic compound, or a chemical compound, or a combination of two or more substances. Unless otherwise specified, the terms “agent,” “candidate agent,” “test agent,” “substance,” and “compound” are used interchangeably.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

The terms “individual,” “host,” “subject,” and “patient,” used interchangeably herein, refer to a mammal, including, but not limited to, murines, felines, simians, humans, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “an agent” includes a plurality of such agents and reference to “the DDAH polypeptide” includes reference to one or more DDAH polypeptides and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of identifying agents that increase dimethylarginine dimethylaminohydrolase (DDAH) levels and/or activity. The present invention further provides agents that increase DDAH levels and/or activity, and compositions comprising the agents. The invention further provides methods of treating insulin resistance, and methods of lowering blood glucose levels in an insulin-resistance individual, the methods generally involving administering to the individual an effective amount of an agent that increases DDAH levels and/or activity. The invention further provides methods of treating other conditions associated with elevated levels of plasma ADMA.

In contrast to reports in the literature that insulin resistance causes increased ADMA levels, the present invention is based in part on the observation that ADMA induces insulin resistance in mice. The present invention is further based in part on the observation that while wild-type mice treated with ADMA exhibit impaired plasma glucose clearance, DDAH overexpressing, ADMA-treated transgenic mice exhibit plasma glucose clearance rates comparable to those of PBS-treated wild-type mice. Thus, in contrast to previous reports, the inventors discovered that increased ADMA levels can lead to insulin resistance, and increasing DDAH levels can overcome ADMA-induced insulin resistance. Based on these observations, the present invention relates to agents and methods for increasing DDAH levels and/or activity.

Furthermore, the inventors have shown for the first time in vivo, that an increase in DDAH expression and activity can reduce plasma ADMA levels, and that this effect is associated with an increase in nitric oxide (NO) synthesis. The increase in NO synthesis is sufficiently great to cause a reduction in systemic resistance and blood pressure.

Screening Methods

The present invention provides methods of identifying agents that modulate DDAH levels and/or activity. Of particular interest in many embodiments are methods of identifying agents that increase DDAH activity and/or levels. Agents that increase DDAH activity and/or levels are expected to lower plasma ADMA levels and to treat insulin resistance.

In some embodiments, the invention provides methods for identifying agents that increase DDAH activity. The methods generally involve contacting a sample comprising DDAH with a test agent; and determining the effect, if any, of the test agent on DDAH activity. Typically, the sample also includes a substrate for DDAH.

As used herein, the term “determining” refers to both quantitative and qualitative determinations and as such, the term “determining” is used interchangeably herein with “assaying,” “measuring,” and the like.

In some embodiments, the invention provides methods for identifying agents that increase a level of DDAH mRNA and/or protein. The methods generally involve contacting a cell that comprises a nucleic acid comprising a nucleotide sequence that encodes DDAH; and determining the effect, if any, of the test agent on DDAH mRNA and/or polypeptide levels.

The terms “candidate agent,” “test agent,” “agent”, “substance” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Assays of the invention include controls, where suitable controls include a sample (e.g., a sample comprising DDAH and substrate in the absence of the test agent; a sample comprising a cell in the absence of test agent; a cell sample in the absence of test agent; etc.). Generally a plurality of assay mixtures is run in parallel with different agent concentrations to obtain a differential response to the various concentrations. Typically, one of these concentrations serves as a negative control, i.e. at zero concentration or below the level of detection.

A variety of other reagents may be included in the screening assay. These include reagents like salts, neutral proteins, e.g. albumin, detergents, etc., including agents that are used to facilitate optimal enzyme activity and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity, but may also be optimized to facilitate rapid high-throughput screening. Typically between 0.1 and 1 hour will be sufficient.

The screening methods may be designed a number of different ways, where a variety of assay configurations and protocols may be employed, as are known in the art. For example, one of the components may be bound to a solid support, and the remaining components contacted with the support bound component. The above components of the method may be combined at substantially the same time or at different times.

DDAH

“DDAH” as used herein is meant to encompass either isoform of dimethylaminohydrolase, DDAH-1 and DDAH-2, as well as enzymatically active fragments of DDAH. The polynucleotide and polypeptide sequences for both isoforms of DDAH are known (see, e.g, GenBank accession nos. NM_{—}012137 and AJ012008 (human DDAH-1); NM_{—}013974 (human DDAH-2); NM_{—}016765, XM_{—}128481, and XM_{—}110196 (mouse DDAH-2); and NM_{—}022297 (rat DDAH-1)). Recombinant DDAH has been expressed, and the DNA and amino acid sequence characterized. For example, DNA and amino acid sequences for human DDAH-1 and human DDAH-2, as well as their characterization and homology to rat DDAH and arginine deaminase of Pseudomonas putida, is described in Leiper et al. (1999) Biochem J343 Pt 1:209-14. DNA and amino acid sequences for human DDAH-1 and human DDAH-2, as well as their characterization, and identification of fragments and fusion proteins that retain DDAH activity, are described in Kimoto et al. (1998) Eur J Biochem 258(2):863-8. “DDAH” as used herein thus also encompasses fragments, fusion proteins, and variants (e.g., variants having one or more amino acid substitutions, addition, deletions, and/or insertions) that retain DDAH enzymatic activity. Specific enzymatically active DDAH variants, fragments, fusion proteins, and the like can be verified by adapting the methods described herein.

DDAH fusion proteins suitable for use in a subject screening method comprise DDAH (or an enzymatically active fragment thereof) and a heterologous polypeptide (a “fusion partner”), where suitable fusion partners include immunological tags such as epitope tags, including, but not limited to, hemagglutinin, FLAG, and the like; proteins that provide for a detectable signal, including, but not limited to, fluorescent proteins (e.g., a green fluorescent protein), chromogenic proteins, enzymes (e.g., β-galactosidase, luciferase, horse radish peroxidase, etc.), and the like; polypeptides that facilitate purification or isolation of the fusion protein, e.g., metal ion binding polypeptides such as 6His tags (e.g., DDAH/6His), glutathione-S-transferase, and the like; polypeptides that provide for subcellular localization; and polypeptides that provide for secretion from a cell.

DDAH used in the assay of the invention can be isolated from a source of the enzyme (e.g., from cells that naturally produce DDAH), by synthetic methods, or by recombinant techniques, each of which methods are well known in the art. “DDAH” as used herein is also meant to encompass polypeptide variants and fragments of DDAH-1 and DDAH-2 that retain enzymatic activity. The amino acid sequences of DDAH are highly conserved between mammalian species. For example, mouse and human DDAH-1 share 98% amino acid identity, while rat and human DDAH-1 share 95% amino acid sequence identity (see, e.g., Leiper et al. (1999) Biochem J. 343:209-214. These similarities in amino acid sequence provide guidance for amino acid residue alterations that can be made to DDAH while retaining enzymatic activity. In addition, the highly conserved nature of the DDAH proteins among mammalian species also indicates that these mammalian DDAH enzymes can be used interchangeably in the screening assays of the invention.

Identifying Agents that Modulate DDAH Enzymatic Activity

In some embodiments, the invention provides methods for identifying agents that increase DDAH activity. In many embodiments, such assays are cell-free in vitro assays. The methods generally involve contacting a sample (e.g., a cell-free sample) comprising DDAH with a test agent in vitro; and determining the effect, if any, of the test agent on DDAH activity. Typically, the sample also includes a substrate for DDAH, e.g., asymmetric dimethylarginine (ADMA). DDAH enzymatic activity can be measured by assessing the effect of DDAH on its substrate ADMA under appropriate conditions. Enzymatic activity is calculated by measuring the concentration of the enzymatic product L-citrulline. Measurement of L-citrulline may be conducted according to the method disclosed in Prescott and Jones (1969) Anal. Biochem. 32,408-419.

To establish a correlation between enzyme activity and amount of enzyme, one unit of enzyme activity may be defined as the amount of enzyme catalyzing the formation of 1 μmol/L of L-citrulline per minute at 37° C.

A test agent of interest is one that increases DDAH activity by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, at least about 2-fold, at least about 5-fold, at least about 10-fold, at least about 20-fold, at least about 50-fold, or at least about 100-fold, or more, when compared to a control in the absence of the test agent.

Identifying Agents that Modulate DDAH mRNA and/or Polypeptide Levels

In some embodiments, the invention provides methods for identifying agents that increase a level of DDAH mRNA and/or DDAH polypeptide. The methods generally involve contacting a cell that comprises a nucleic acid comprising a nucleotide sequence that encodes DDAH; and determining the effect, if any, of the test agent on DDAH mRNA and/or polypeptide levels. In many embodiments, the assay is an in vitro, cell-based assay.

A candidate agent is assessed for any cytotoxic activity it may exhibit toward the cell used in the assay, using well-known assays, such as trypan blue dye exclusion, an MTT (3-(4,5-dimethylthiazol-2-yl)-2,5-diphenyl-2H-tetrazolium bromide) assay, and the like. Agents that do not exhibit cytotoxic activity are considered candidate agents.

A wide variety of cell-based assays may be used for identifying agents which increase a level of DDAH mRNA in a eukaryotic cell, using, for example, a eukaryotic that normally produces DDAH mRNA, a mammalian cell transformed with a construct comprising a DDAH-encoding cDNA such that the cDNA is overexpressed, or, alternatively, a cell genetically modified with a construct comprising a DDAH promoter operably linked to a reporter gene. Where the assay is an in vitro cell-based assay, any of a variety of cells can be used.

The cells used in the assay are usually eukaryotic cells, including, but not limited to, rodent cells, human cells, and yeast cells. The cells may be primary cell cultures or may be immortalized cell lines. The cells may be “recombinant,” e.g., the cell may have transiently or stably introduced therein a construct (e.g., a plasmid, a recombinant viral vector, or any other suitable vector) that comprises a nucleotide sequence encoding a DDAH polypeptide, or that comprises a nucleotide sequence that comprises a DDAH promoter operably linked to a reporter gene.

Accordingly, the present invention provides a method for identifying an agent, particularly a biologically active agent, that increases a level of DDAH expression in a cell, the method comprising: combining a candidate agent to be tested with a cell comprising a nucleic acid which encodes a DDAH polypeptide, or a construct comprising a DDAH promoter operably linked to a reporter gene; and determining the effect of said agent on DDAH expression. An increase of at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, in the level (i.e., an amount) of DDAH mRNA and/or polypeptide following contacting the cell with a candidate agent being tested, compared to a control to which no agent is added, indicates that the agent increases DDAH expression.

DDAH mRNA and/or polypeptide whose levels are being measured can be encoded by an endogenous DDAH polynucleotide, or the DDAH polynucleotide encoding the DDAH mRNA and/or polypeptide can be one that is comprised within a recombinant vector and introduced into the cell, i.e., the DDAH mRNA and/or polypeptide can be encoded by an exogenous DDAH polynucleotide. For example, a recombinant vector may comprise a DDAH transcriptional regulatory sequence, such as a promoter sequence, operably linked to a DDAH coding region.

Alternatively, in some embodiments, a recombinant vector may comprise a DDAH transcriptional regulatory sequence operably linked to a reporter gene (e.g, β-galactosidase, chloramphenicol acetyl transferase, a fluorescent protein, luciferase, or other gene that can be easily assayed for expression), and the level of the reporter gene can be assayed. In these embodiments, the method for identifying an agent that modulates a level of DDAH expression in a cell, comprises: combining a candidate agent to be tested with a cell comprising a nucleic acid (e.g., an exogenous nucleic acid) which comprises a DDAH gene transcriptional regulatory element operably linked to a reporter gene; and determining the effect of said agent on reporter gene expression.

A recombinant vector may comprise an isolated DDAH transcriptional regulatory sequence, such as a promoter sequence, operably linked to sequences coding for a DDAH polypeptide; or the transcriptional control sequences can be operably linked to coding sequences for a DDAH fusion protein comprising DDAH polypeptide fused to a polypeptide which facilitates detection. In these embodiments, the method comprises combining a candidate agent to be tested with a cell comprising a nucleic acid which comprises a DDAH gene transcriptional regulatory element operably linked to a DDAH polypeptide-coding sequence; and determining the effect of said agent on DDAH expression, which determination can be carried out by measuring an amount of DDAH mRNA, DDAH polypeptide, or DDAH fusion polypeptide produced by the cell.

Cell-based in vitro assays generally comprise the steps of contacting the cell with an agent to be tested, forming a test sample, and, after a suitable time, assessing the effect of the agent on DDAH expression. A control sample comprises the same cell without the candidate agent added. DDAH expression levels are measured in both the test sample and the control sample. A comparison is made between DDAH expression level in the test sample and the control sample. DDAH expression can be assessed using conventional assays. For example, when a mammalian cell line is transformed with a construct that results in expression of DDAH, DDAH mRNA levels can be detected and measured, or DDAH polypeptide levels can be detected and measured. A suitable period of time for contacting the agent with the cell can be determined empirically, and is generally a time sufficient to allow entry of the agent into the cell and to allow the agent to have a measurable effect on DDAH mRNA and/or polypeptide levels. Generally, a suitable time is between 10 minutes and 24 hours, or from about 1 hour to about 8 hours.

Detecting DDAH mRNA levels

Methods of measuring DDAH mRNA levels are known in the art, and any of these methods can be used in the methods of the present invention to identify an agent which modulates DDAH mRNA level in a cell, including, but not limited to, a polymerase chain reaction (PCR), such as a PCR employing detectably labeled oligonucleotide primers, and any of a variety of hybridization assays. The DDAH mRNA may be assayed directly or reverse transcribed into cDNA for analysis. The mRNA may be purified, but need not be. The mRNA is in some embodiments isolated from the cell. In other embodiments, quantitation is performed on a cell lysate.

DDAH mRNA may be amplified by conventional techniques, such as a PCR method, to provide sufficient amounts for analysis. The use of PCR is described in Saiki, et al. (1985), Science 239:487, and a review of techniques may be found in Sambrook, et al. Molecular Cloning: A Laboratory Manual, CSH Press 1989, pp. 14.2-14.33. Quantitative PCR techniques are amply described in the literature, and are suitable for use in a subject method. See, e.g., Quantitative PCR Protocols (Methods in Molecular Medicine, Vol. 26), B. Kochanowski and U. Reischl, eds., (1999) Humana Press; and The PCT Technique: Quantitative PCR (BioTechniques Update series), J. W. Larrick, ed. (1997) Eaton Publ. Co.

A detectable label may be included in an amplification reaction, e.g., a PCR reaction. Suitable labels include fluorochromes, e.g fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The following is an exemplary, non-limiting example of a PCR reaction that would be suitable for use in a subject method. A PCR reaction mixture is set up that contains the following components: DNA 100 ng; 10× Buffer (100 mM Tris, pH 8.3; 500 mM KCl) δ 1; 25 mM dNTPs 4 μl; 25 mM MgCl₂ 3 i; Forward Primer (10 μM) 2 μl; Reverse Primer (10 μM) 2 μl; Taq polymerase (5 U/μl) 0.2 μl; volume total 50 μl. Suitable PCR parameters are as follows: 95° C. 3 minutes to denature the template; followed by 35 cycles of: 94° C. 1 minute; 55° C. 1 minute; and 72° C. 1.5 minute. Following the 35 cycles, the reaction is carried out further at 72° C. 10 minutes. Those skilled in the art can readily determine other suitable PCR components and conditions. Suitable primers are readily designed based on available nucleotide sequences encoding DDAH polypeptides.

A variety of other methods for determining the nucleic acid abundance in a sample are known to those of skill in the art, where particular methods of interest include those described in: Pietu et al., Genome Res. (June 1996) δ: 492-503; Zhao et al., Gene (Apr. 24, 1995) 156: 207-213; Soares, Curr. Opin. Biotechnol. (October 1997) δ: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

Detecting DDAH Polypeptide Levels

Similarly, DDAH polypeptide levels can be measured using any standard method, several of which have been described herein, including, but not limited to, an immunoassay such as an enzyme linked immunosorbent assay (ELISA), for example an ELISA employing a detectably labeled antibody specific for a DDAH polypeptide.

DDAH polypeptide levels can also be measured in cells harboring a recombinant construct comprising a nucleotide sequence that encodes a DDAH fusion protein, where the fusion partner provides for a detectable signal or can otherwise be detected. For example, where the fusion partner provides an immunologically recognizable epitope (an “epitope tag”), an antibody specific for an epitope of the fusion partner can be used to detect and quantitate the level of DDAH. In some embodiments, the fusion partner provides for a detectable signal, and in these embodiments, the detection method is chosen based on the type of signal generated by the fusion partner. For example, where the fusion partner is a fluorescent protein, fluorescence is measured. Fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Philosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; and the like. Where the fusion partner is an enzyme that yields a detectable product, the product can be detected using an appropriate means, e.g., β-galactosidase can, depending on the substrate, yield colored product, which is detected spectrophotometrically, or a fluorescent product; luciferase can yield a luminescent product detectable with a luminometer; etc.

A number of methods are available for determining the level of a protein in a particular sample. For example, detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. Alternatively, the secondary antibody conjugated to a fluorescent compound, e.g. fluorescein, rhodamine, Texas red, etc. The absence or presence of antibody binding may be determined by various methods, including flow cytometry of dissociated cells, microscopy, radiography, scintillation counting, etc.

Agents

The present invention further provides active agents that increase a level and/or activity of DDAH in a cell of an individual. The agents are useful for treating insulin resistance; and for reducing plasma glucose levels in an individual. The agents are also useful for reducing plasma or tissue levels of ADMA in conditions characterized by elevated levels of ADMA, such as hypercholesterolemia, hypertension, hyperglycemia, hyperhomocysteinemia, and atherosclerosis. The present invention further provides compositions, including pharmaceutical compositions, comprising a subject agent.

In some embodiments, an “active” agent is an agent that increases a level and/or activity of DDAH, and that is effective to reduce a blood glucose level by at least about 10%, at least about 15%, at least about 20%, at least about 25%, at least about 30%, at least about 40%, or at least about 50% when compared to the blood glucose levels in the absence of the active agent. In some embodiments, an “active” agent is effective to reduce blood glucose levels to a normal range. Normal blood glucose levels are typically in the range of from about 70 mg/dL to about 110 mg/dL before a meal (e.g., a fasting blood glucose level); and less than 120 mg/dL 2 hours after a meal.

In some embodiments, an “active” agent is an agent that increases a level and/or activity of DDAH, and that is effective to reduce a plasma or tissue ADMA level by at least about 10%, at least about 15%, at least about 20%, at least about 25%, at least about 30%, at least about 40%, or at least about 50% when compared to the ADMA levels in the absence of the active agent. In some embodiments, an “active” agent is effective to reduce ADMA levels to a normal range. Normal plasma ADMA levels are generally in the range of from about 0.5 μM to about 1.0 μM before a meal (e.g., a fasting plasma ADMA level).

In many embodiments, the agent is a small molecule, e.g., a small organic or inorganic compound having a molecular weight of more than 50 and less than about 2,500 daltons. Agents may comprise functional groups necessary for structural interaction with proteins, particularly hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Agents are also found among biomolecules including peptides, saccharides, fatty acids, steroids, purines, pyrimidines, derivatives, structural analogs or combinations thereof.

In some embodiments, a subject agent is formulated with one or more pharmaceutically acceptable excipients. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Formulations, Dosages, and Routes Of Administration

The invention provides formulations, including pharmaceutical formulations, comprising an active agent that increases a level and/or an activity of DDAH. In general, a formulation comprises an effective amount of an agent that increases a level and/or an activity of DDAH. An “effective amount” means a dosage sufficient to produce a desired result, e.g., an increase in a level and/or an activity of DDAH, a reduction in insulin resistance, a reduction in plasma glucose levels, a reduction in plasma ADMA levels. Generally, the desired result is at least an increase in a level and/or an activity of DDAH as compared to a control.

Formulations

In the subject methods, the active agent(s) may be administered to the host using any convenient means capable of resulting in the desired increase in a level and/or an activity of DDAH. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Other modes of administration will also find use with the subject invention. For instance, an agent of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

In some embodiments, a subject agent is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. In some embodiments, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are particularly preferred due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a subject agent is delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Dosages

Although the dosage used will vary depending on the clinical goals to be achieved, a suitable dosage range is one which provides up to about 1 μg to about 1,000 μg or about 10,000 μg of an agent that increases a level and/or an activity of DDAH can be administered in a single dose. Alternatively, a target dose of an agent that increases a level and/or an activity of DDAH can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of Administration

An agent that increases a level and/or an activity of DDAH is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, rectal, nasal, oral and other enteral and parenteral routes of administration. Routes of administration may be combined, if desired, or adjusted depending upon the agent and/or the desired effect. The composition can be administered in a single dose or in multiple doses.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrastemal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits with unit doses of the active agent, e.g. in oral or injectable doses, are provided. In such kits, in addition to the containers containing the unit doses will be an informational package insert describing the use and attendant benefits of the drugs in treating pathological condition of interest. Preferred compounds and unit doses are those described herein above.

Treatment Methods

The present invention provides methods of treating insulin resistance in an individual; and methods of reducing blood glucose levels in an individual. The methods generally involve administering to an individual in need thereof an effective amount of an active agent that increases a level and/or activity of DDAH.

The present invention also provides methods of reducing high plasma or tissue levels of ADMA in an individual; methods of reducing ADMA levels in an individual; and methods of treating a disorder associated with or caused by elevated plasma or tissue levels of ADMA. The methods generally involve administering to an individual in need thereof an effective amount of an active agent that increases a level and/or activity of DDAH.

In some embodiments, an active agent is administered following a meal, e.g., within 2 hours after a meal, e.g., from about 1 minute to about 2 hours after a meal. In other embodiments, an active agent is administered before a meal, e.g., from about 1 minute to about 30 minutes before a meal. In other embodiments, an active agent is administered as needed to lower blood glucose levels, e.g., an active agent is administered within about 1 minute to about 30 minutes following a blood glucose measurement that indicates that the blood glucose level exceeds the normal range. In other embodiments, an active agent is administered continuously.

Combination Therapy

In some embodiments, a subject agent is administered in combination therapy with at least a second therapeutic agent. In some embodiments, the second therapeutic agent is an agent that reduces blood glucose levels. In other embodiments, the second therapeutic agent is one that treats a condition that is secondary to insulin resistance, or a condition that is a sequelae of insulin resistance. Suitable agents that can be administered in combination therapy with a subject agent include, but are not limited to, thiazolidinediones, e.g., Avandia® (rosiglitazone maleate); agents of the sulfonylurea class; biguanides, e.g. metformin (Glucophage®), troglitazone (Rezulin®); alpha-glucosidase inhibitors, e.g., acarbose (Precose®); and the like.

Subjects Suitable for Treatment

Subjects suitable for treatment with an agent or method of the present invention include individuals who have been diagnosed with diabetes mellitus. Such individuals include those having a fasting blood glucose level greater than about 126 mg/dL. Such individuals include those having blood glucose levels of greater than about 200 mg/dL following a two-hour glucose tolerance test (75 g anhydrous glucose orally). Also suitable for treatment with an agent or method of the present invention are individuals with metabolic syndrome (which consists of obesity, hypertension, dyslipidemia) and insulin resistance. Also suitable for treatment with a subject agent or a subject method are individuals with a fasting blood insulin level of greater than about 15 μU/ml.

Subjects suitable for treatment also include individuals having a disease or disorder associated with or caused by elevated ADMA levels, including, but not limited to, hypercholesterolemia, hypertension, hyperhomocysteinemia, coronary artery disease, peripheral arterial disease, and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s, second(s); min, minute(s); hr, hour(s); and the like.

### Example 1

### Transgenic Expression of Human DDAH Increases DDAH Activity in Mice, and Reduces Plasma ADMA Levels

This Example shows that DDAH transgenic mice manifest a) increased DDAH expression, as assessed by Western analysis, and b) increased tissue DDAH activity (in skeletal muscle in this example), which is further reflected by a reduction in plasma ADMA levels.

In brief, C57BL/6J mice were used to create the transgenic line. A human DDAH I transgenic expression construct was prepared using human DDAH I cDNA, a human β-actin promoter and RNA processing signals from SV40 derived from a modified human agouti expression vector. These sequences and 700 bp of pBluescript vector were then excised as a 6.9 kb linear fragment, separated by 1.0% agarose gel electrophoresis and eluted using a gel extraction kit. Fertilized eggs were harvested from superovulated C57BL/6J mice for pronuclear injection of purified DNA. Embryos were transferred into the oviducts of pseudopregnant B6CBAF1 foster mothers. Genomic DNA was isolated from tail biopsies at 3 to 4 weeks of age using a DNeasy kit and subjected to Southern blot analysis to identify the transgene. Southern blots were interrogated with a ³²P -labeled probe to the Bam-HI fragment of human DDAH I cDNA. Genomic DNA digested by Sal-I and Eco-R1 yielded a single band of the expected size in transgenic mice only. Heterozygous transgenic mice were compared to age, weight and sex matched wild type littermates.

Western analysis was performed to quantitate DDAH expression levels in murine tissues. Plasma and tissue concentrations of L-arginine, ADMA and symmetric dimethylarginine (SDMA) were measured by HPLC and precolumn derivatization with o-phthaldialdehyde. DDAH enzyme activity was assayed by determining citrulline formation from ADMA.

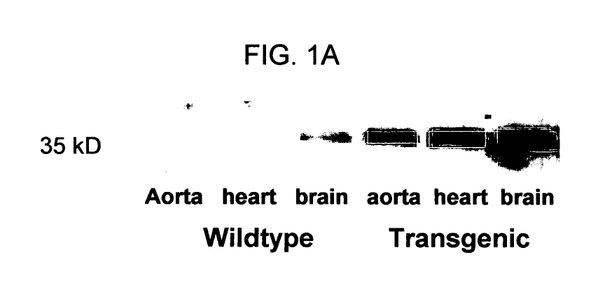

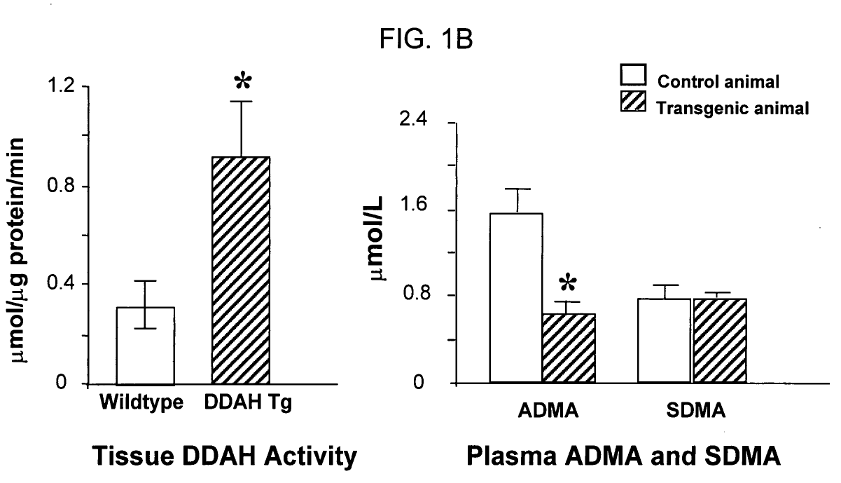

As shown in FIG. 1A, elevated levels of human DDAH protein are expressed in various tissues (aorta, heart, brain) of the DDAH transgenic mouse. As shown in FIG. 1B, elevated DDAH activity was detected in tissues of the transgenic mouse. Elevated DDAH activity is associated with a reduction in the plasma ADMA level.

### Example 2

### DDAH Transgenic Mice Manifest Higher Concentrations of Urinary NOₓ Indicating Increased Endogenous NO Synthesis

Briefly, transgenic and control mice were placed singly in metabolic cages for urine collection. Urine was collected into plastic vials containing isopropanol, cooled by ice immersion during the 12 hr collection period. To eliminate dietary sources of nitrogen oxides (NOₓ), animals were fasted, and received nitrate-free water containing 10% dextrose to support caloric needs. Urine was centrifuged and the supernatant assayed for NOₓ using a commercially available fluorimetric assay.

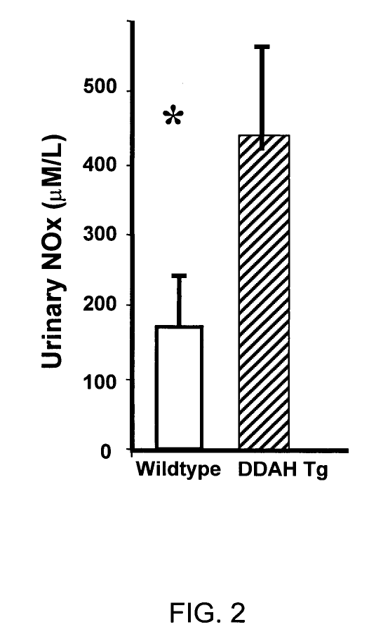

As shown in FIG. 2, elevated levels of urinary nitrogen oxides (NOₓ) were detected in the DDAH transgenic mouse, revealing that increased DDAH activity is associated with increased NO synthesis.

### Example 3

### DDAH Transgenic Mice Manifest an Increased Heart Rate (HR), with Mean Arterial Pressure (MAP) and Systemic Vascular Resistance (SVR) Tending to be Decreased by about 10%

The cardiac output (CO) in the transgenic animals was similar to that of the control animals (20±2.3 ml/mᵢn vs. 21±3.1 ml/min). Briefly, systolic, diastolic, and mean arterial blood pressure were measured in anesthetized male mice using 1.4 F ultraminiature pressure catheter inserted through the carotid artery. Pressure waveforms were digitized and analyzed using a Powerlab data recording system.

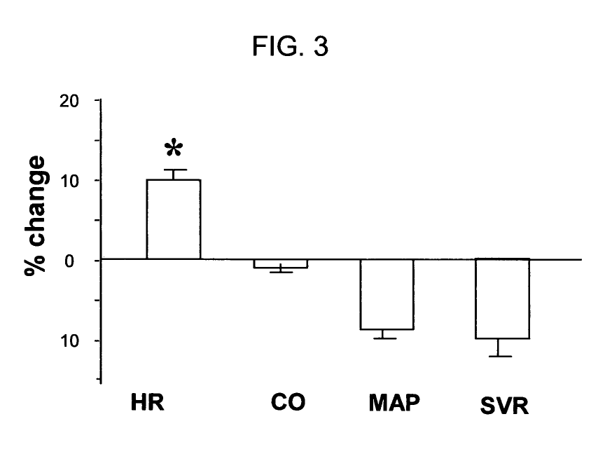

As shown in FIG. 3. DDAH transgenic mice manifest an increased heart rate, with MAP and SVR tending to be decreased by about 10%. The reduction in MAP and SVR in the DDAH transgenic mouse is presumably due to an increased elaboration of NO in these animals. In addition, the cardiac output is not increased, probably because of the negative inotropic effect of NO in the heart.

### Example 4

### DDAH Transgenic Mice are Insulin Sensitive, and Resistant to Adverse Effects of ADMA

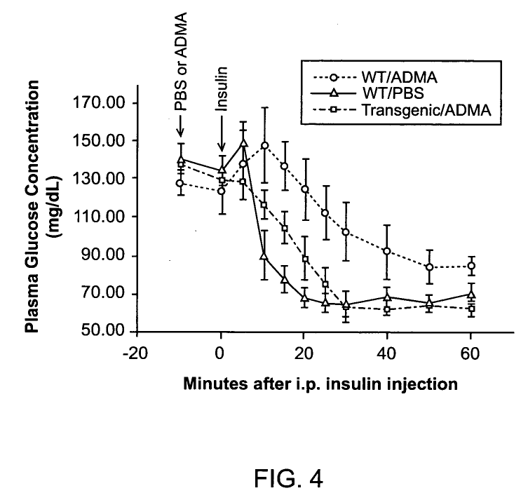

Experiments were performed to assess insulin sensitivity by measuring plasma glucose clearance rate in these DDAH transgenic mice. After 4 hours of fasting, wildtype female mice were injected intraperitoneally with either ADMA (75 mg/kg), which induces insulin resistance, or phosphate buffered saline (PBS); the same dosage was given to the transgenic mice (n=3 in each group). After two hours, insulin was injected; venous blood glucose was assayed by a glucometer every 5 minutes for an hour. The half-life of glucose disappearance from plasma was calculated using a first-order decay model; plasma ADMA was measured by HPLC. As shown in FIG. 4, wild-type, ADMA-treated mice exhibited a delayed and attenuated drop in plasma glucose in response to insulin, compared to PBS-treated wild-type mice. ADMA-treated DDAH overexpressing transgenic mice exhibited plasma glucose clearance that was comparable to PBS-treated wild-type mice. These results show that overexpression of DDAH reduces the effect of ADMA in inducing insulin resistance.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An in vitro method of identifying an agent that increases enzymatic activity of dimethylarginine dimethylaminohydrolase (DDAH), the method comprising:
a) contacting DDAH with a substrate for DDAH; and a test agent; and
b) determining the effect, if any, of the test agent on DDAH enzymatic activity.

2. The method of claim 1, wherein said determining step comprises measuring the level of L-citrulline produced with asymmetric dimethylarginine as substrate.

3. The method of claim 2, wherein said measuring comprises use of a colorimetric assay.

4. The method of claim 1, wherein the DDAH is DDAH-2.

5. The method of claim 1, wherein the DDAH is human DDAH.

6. An in vitro method of identifying an agent that increases the level of dimethylarginine dimethylaminohydrolase (DDAH) polypeptide in a cell, the method comprising:
a) contacting a cell that produces DDAH with a test agent; and
b) determining the effect, if any, of the test agent on the level of DDAH polypeptide in the cell.

7. The method of claim 6, wherein said determining comprises an immunological assay with an antibody specific for DDAH.

8. The method of claim 7, wherein said immunological assay is an enzyme linked immunosorbent assay.

9. The method of claim 7, wherein said antibody is detectably labeled.

10. The method of claim 6, wherein the DDAH polypeptide produced by the cell is a fusion protein comprising DDAH and a fusion partner.

11. An in vitro method of identifying an agent that increases the level of a dimethylarginine dimethylaminohydrolase (DDAH) mRNA in a cell, the method comprising:
a) contacting a cell that produces a DDAH mRNA with a test agent; and
b) determining the effect, if any, of the test agent on the level of DDAH mRNA in the cell.

12. The method of claim 11, wherein said determining step comprises a polymerase chain reaction (PCR) assay to measure a level of DDAH mRNA in the cell.

13. The method of claim 11, wherein the cell is one that does not produce endogenous DDAH mRNA, and wherein the cell is genetically modified to express exogenous DDAH mRNA.

14. The method of claim 13, wherein the cell is genetically modified with a construct that comprises a DDAH promoter operably linked to a reporter gene, and wherein said determining step comprises measuring the level of reporter gene expression.

15. The method of claim 14, wherein said reporter gene encodes an enzyme, and wherein said determining step comprises measuring the level of enzyme encoded by the reporter gene.

16. An agent identified by the method according to any one of claims 1, 6, and 11.

17. A composition comprising the agent according to claim 16.

18. The composition of claim 17, wherein said composition comprises a pharmaceutically acceptable excipient.

19. The use of an agent identified according to any one of the claims 1, 6 and 11 in the treatment of conditions characterized by insulin resistance.

20. The use of an agent identified according to any one of the claims 1, 6 and 11 in the treatment of conditions characterized by elevated levels of plasma or tissue ADMA.
